(19) United States
(12) Patent Application Publication
(10) Pub. No.: US 2014/0200577 A1
(43) Pub. Date: Jul. 17, 2014

(54) ABLATION GRASPER

(71) Applicants: George Klein, London (CA); Harold Wodlinger, Thornhill (CA); Steve Wayne Berhow, St. Michael, MN (US); Douglas Scott Wahnschaffe, Monticello, MN (US)

(72) Inventors: George Klein, London (CA); Harold Wodlinger, Thornhill (CA); Steve Wayne Berhow, St. Michael, MN (US); Douglas Scott Wahnschaffe, Monticello, MN (US)

(21) Appl. No.: 14/115,595

(22) PCT Filed: May 3, 2012
(86) PCT No.: PCT/CA12/00420

§ 371 Date: Mar. 21, 2014

Related U.S. Application Data
(60) Provisional application No. 61/482,408, filed on May 4, 2011.

(51) Int. Cl. A61B 18/14

(52) U.S. Cl. CPC A61B 18/1492; A61B 18/1445
USPC 606/41

(57) ABSTRACT

The present invention provides improved catheters for ablative procedures for biological tissue, e.g., in the heart. The catheters allow active fixation to the tissue using a pair of jaws, and a sheath actuates the jaws, simplifying actuation. In particular embodiments, the pair of jaws provides additional stability in attachment to difficult ablation sites, e.g., along the ridges of cardiac structures.

### CROSS-REFERENCE TO RELATED APPLICATIONS

This application claims benefit of U.S. provisional application No. 61/482,408, filed May 4, 2011, which is hereby incorporated by reference.

### BACKGROUND OF THE INVENTION

The invention relates to the fields of medical devices and methods of their use.

Ablation catheters generally consist of tubular structures with circumferential electrodes placed along the shaft and at the tip. These catheters also include a pull-wire or other steering mechanism that allows the physician to manipulate the angle of the tip with respect to the shaft of the catheter. Typically, the tip of the catheter is positioned under fluoroscopic or non-fluoroscopic control against the target of interest. To maintain the tip's position while the heart is beating, a steering mechanism guides the catheter and exerts torque on the tip against the myocardial tissue to optimize effective contact. In most cases, this is adequate in order to maintain proper contact during ablation. However, maintaining contact with some cardiac structures, in particular along ridges, is extremely challenging. Ablation near these structures often fails because the ablation catheter slips away from the site after the ablation has started.

Accordingly, there is a need for new catheters capable of maintaining proper contact with biological tissues during ablation.

### SUMMARY OF THE INVENTION

In general, the invention provides improved catheters that are actively fixated to biological tissue and will not slip or lose contact during an ablation procedure. In particular, this catheter is simple to manufacture using currently available proven technology.

In one aspect, the invention features a device for tissue ablation including a catheter having a proximal and distal end; a pair of jaws comprising an ablation electrode disposed at the distal end of the catheter; and a sheath movable with respect to the catheter, wherein, when the sheath is disposed proximally, the pair of jaws is biased to an open position, and, when the sheath is disposed distally, the pair of jaws is biased to a closed position. The catheter or sheath can further include an irrigation lumen and a port for the release of irrigating fluid at the distal end. Such a port can be disposed, e.g., in the pair of jaws or proximal to the pair of jaws. In certain embodiments, the sheath or pair of jaws can further include mapping electrodes. In certain embodiments, the pair of jaws is the ablation electrode. The device can further include a mechanical element, e.g., a spring, to bias the pair of jaws open when the sheath is disposed proximally. In other embodiments, the pair of jaws is shaped to be biased open when the sheath is disposed proximally, e.g., where the pair of jaws is shaped from a single piece of flexible material. In other embodiments, the pair of jaws further includes a return electrode. The distal end of said catheter and/or sheath can be deflectable. The device can further include a lock that maintains the jaws in a closed state when the sheath is disposed proximally, wherein releasing the lock when the sheath is disposed distally results in opening of the jaws.

In another aspect, the invention provides a method of ablating a tissue by inserting any device of the invention into a subject, e.g., a human patient; placing the pair of jaws adjacent to a tissue of interest; actuating the pair of jaws by moving the sheath to grasp the tissue of interest; and actuating the ablation electrode to ablate the tissue. Ablation may be unipolar or bipolar as described herein. In the methods of the invention, a device described herein may be inserted into any appropriate lumen. Exemplary lumens include intravascular spaces and spaces within organs (e.g., the heart, lungs and/or bronchi, stomach, rectum, and urinary bladder). An exemplary tissue of interest is in the heart, e.g., along a ridge of a cardiac structure. The method may further include irrigating the tissue of interest, e.g., through a port in the device or by a separate irrigation source. For devices in which the pair of jaws is locked, the methods may include the step of unlocking the jaws prior to grasping the tissue. Positioning of the device may also include actuating a deflectable catheter and/or sheath, when present in the device.

Exemplary size, lengths, and uses of devices of the invention are provided herein.

Other features and advantages will be apparent from the following description, the drawings, and the claims.

By “subject” is meant any animal, e.g., a human, other primate, other mammal, a bird, a reptile, or an amphibian.

### BRIEF DESCRIPTION OF THE DRAWINGS

FIGS. 1A-1E are schematic depictions of an exemplary intraluminal catheter having a pair of jaws with flexible material construction. FIGS. 1A and 1B are top and side views of the catheter and sheath, which illustrate that the jaws remain open without the sheath. FIGS. 1C and 1D are side and end views of the pair of jaws, and FIG. 1E is a depiction of the open, half closed, and closed positions of the pair of jaws.

FIGS. 2A-2D are schematic depictions of another exemplary intraluminal catheter having a pair of jaws biased by a spring. FIGS. 2A and 2B are top and side views of the catheter and sheath, which illustrate that the jaws remain open without the sheath. FIG. 2C is a set of end views of the pair of jaws, and FIG. 2D is a depiction of the open, half closed, and closed positions of the pair of jaws.

FIGS. 3A-3C are schematic depictions of the use of a device of the invention to map, grasp, and ablate tissue.

### DETAILED DESCRIPTION OF THE INVENTION

The present invention provides improved catheters for ablative procedures for biological tissue, e.g., in the heart. The catheters allow active fixation to the tissue using a pair of jaws, and a sheath actuates the jaws, simplifying actuation. In particular embodiments, the pair of jaws provides additional stability in attachment to difficult ablation sites, e.g., along the ridges of cardiac structures.

### Device

The device includes an ablation catheter with jaws at the distal end. The jaws include or act as at least one ablating electrode. The jaws are biased into an open state and actuated by a sheath that slides over the catheter and mechanically compresses the jaws. Importantly, the device does not employ a pull wire to actuate the jaws. An advantage of this method is that grip force can be applied in a graded fashion by both pushing the catheter and progressively sliding the sheath over the catheter.

As discussed above, the jaws are biased in an open state, so that in the absence of force from the sheath, the jaws do not close or grasp. The jaws may be biased open by any suitable mechanism. Exemplary mechanisms include the use of a spring-loaded hinge or fabrication of the jaws in an open state from a material having a mechanical memory, e.g., a metal such as a shape memory alloy. In one embodiment, the pair of jaws is constructed from a single piece of a flexible material, where actuation of a sheath over the single piece results in folding. The jaws may be smooth or have texture to aid in gripping a tissue. Any texture may be present on only a portion of one or both of the jaws. For example, the jaws may be serrated, grooved, or have a hook-like end or series of bumps. Typically, any texture on the jaws will not have a surface for cutting of tissue. One or both of the pair of jaws may be made of a metal or other conductive material and act as an ablation electrode. Alternatively, an electrode is integrated into one or both of the pair of jaws. Materials for electrodes are known in the art (see, e.g., U.S. Pat. No. 5,916,213).

In one embodiment, the jaws stay closed in the sheath and open when the sheath is moved sufficiently proximally. In a second embodiment, the jaws are locked together even when the sheath is moved proximally. In this embodiment, the locked jaws could be used as a conventional ablation catheter until grasping is required. Various mechanisms may be used to lock and unlock the jaws, including a magnetic coupling, a mechanical lock controlled from the catheter handle, or an unlocking mechanism located at a distance proximal to the jaws that is activated by the sheath. Once the jaws are unlocked, the sheath would be used to close the jaws by sliding the sheath distally as described above. The catheter could also be used with the jaws in the open position to extend the effective electrode surface area in contact with muscle, e.g., for facilitating ablation lines.

Ablation catheters are known in the art (e.g., U.S. Pat. No. 5,916,213) and can be adapted to include jaws having or being an ablation electrode. The electrical connections required to activate the ablation electrodes will typically be located within the catheter, e.g., in a lumen or on the surface. The catheter may also include additional recording electrodes mounted on its shaft. For example, one or more sensors, e.g., mapping electrodes (see, e.g., U.S. Pat. No. 4,960,134) or pressure or temperature transducers (see, e.g., US 2008/0275367), may be positioned at the distal portion of the catheter. Electrodes allow the measurement of electrograms in order to confirm correct placement of the catheter.

It could also contain a lumen for the delivery of fluids to the site of ablation or to the electrodes, e.g., as a cooling mechanism for the ablation electrodes using the delivery of sterile saline. Other fluids that can be delivered include imaging agents and pharmaceutical agents. Ablation is controlled by the user with controls attached to the proximal end of the catheter or a separate voltage source electrically coupled to the catheter. Exit ports for lumens can be at or near the distal end of the catheter and may or may not be present in the jaws themselves, as shown herein.

Sheaths for use with ablation catheters are known in the art. Such sheaths can be adapted to allow for controlled movement of the sheath relative to the jaws to control the gripping force exerted by the jaws. Preferably, the sheath allows for the removal of a particular ablation catheter and replacement with another ablation catheter. Sheaths of the invention may also include a lock to prevent axial and/or longitudinal movement of the catheter relative to the sheath. Exemplary locks include a tab or slot that mates with a corresponding tab or slot on a catheter. Another lock is a clamp capable of applying radial pressure to a catheter. Such a lock may have a high degree of static friction between the sheath and the catheter, e.g., via a detent. The sheath may also include fiducial marks that show the axial position of the sheath relative to the catheter. As with the catheter, the sheath could also include electrodes or other sensors. The sheath may also include one or more lumens for the delivery of fluids at or near the location of the jaws.

Either the ablation catheter or the sheath could be steerable. For example, the sheath may be fixed curve or variably deflectable, and the catheter may be fixed curve or variably deflectable, e.g., as described in U.S. Pat. Nos. 4,601,705, 4,960,134, 6,066,126, and 2005/0267462. If both are steerable, the combination could be used to deflect the tip of the catheter in multiple planes to enhance its versatility. The catheter or sheath will also either include any additional elements required to control the motion or use of the device or be attached to an external component for control of the device.

### Methods

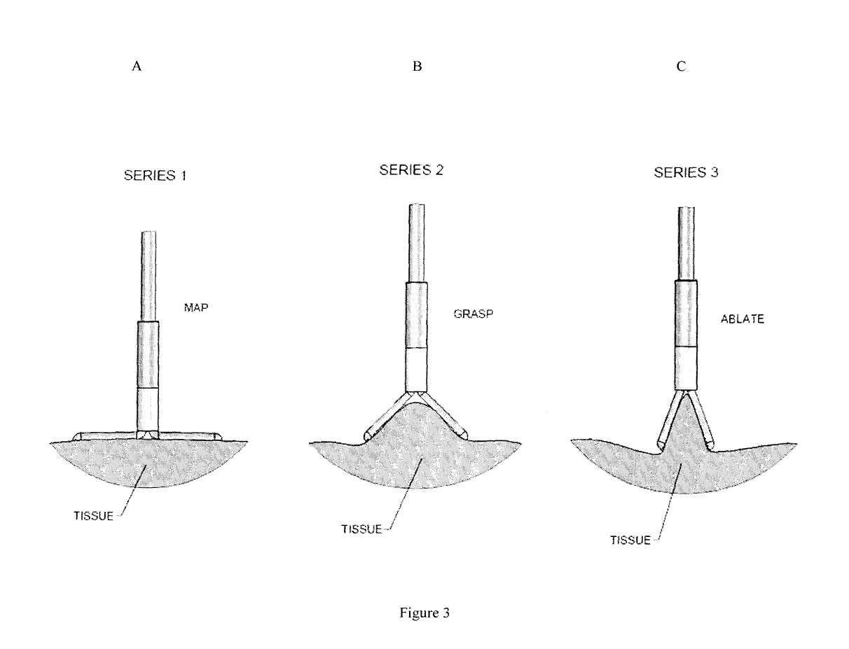

In use, the physician guides the distal end of the catheter within the sheath to the desired ablation site under fluoroscopic, mapping system, magnetic, or other control. The physician would then move the sheath backwards to open the jaws, which are biased open. The sheath could then be moved forward to tighten the jaws progressively to allow the desired grabbing force/contact of the jaws on the tissue to be ablated (FIG. 3). The ablation tip would then move with the myocardium during the heart cycle and would not slip or dislodge during the ablation.

Ablation is performed in either a unipolar or bipolar fashion. In a unipolar fashion, the ablation energy is delivered between an electrode or group of electrodes and a return electrode positioned on the skin of the patient. In a bipolar fashion, the ablation energy is delivered between two different electrodes on the same catheter, for example using an electrode on the upper jaw portion as the ablation electrode, and an electrode on the lower jaw portion as the return electrode.

The devices described herein may be inserted into any appropriate lumen. Exemplary lumens include intravascular spaces and spaces within organs (e.g., the heart, lungs and/or bronchi, stomach, rectum, and urinary bladder). The intended use of the sheath will be used to determine the overall dimensions, the number and position of exits for catheters, and the materials employed in its manufacture, all of which are well known in the art. Typically, a sheath may accommodate catheters and other instruments having diameters between 3 and 34 French, e.g., 4-16 French. A preferred catheter diameter is about 4 mm, with a corresponding lumen diameter of about 5-6 mm. The overall length of the sheath is typically between 10 and 100 cm. In a preferred embodiment, the sheath is sized for percutaneous access to the interior of a human heart or sized for access to the epicardium via an introducer of 10 gauge or smaller diameter.

### EXAMPLE 1

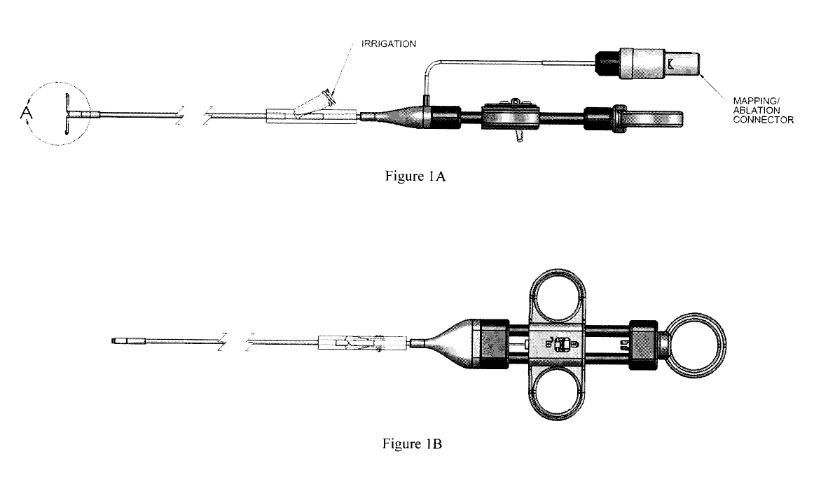

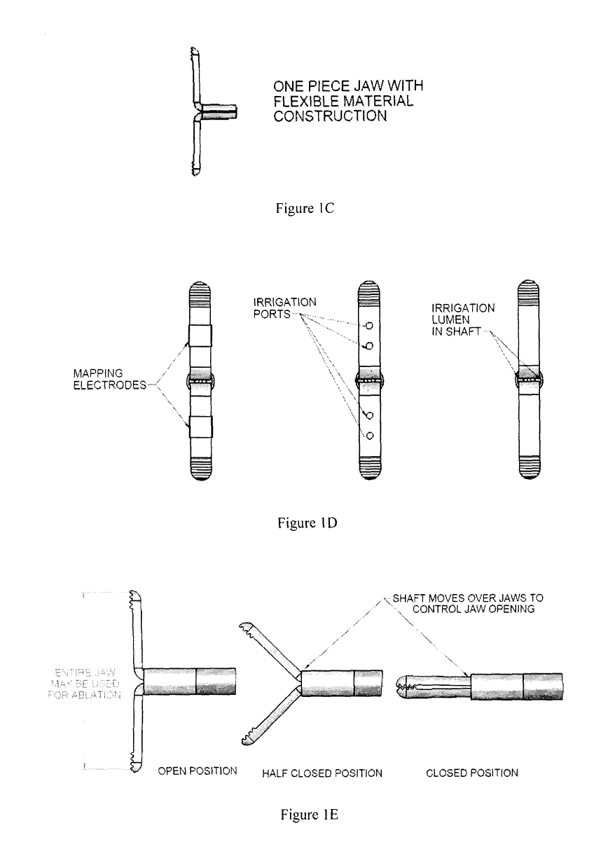

A specific device of the invention is shown in FIGS. 1A-1E. As shown in FIG. 1A, the device includes a catheter disposed within a sheath. The sheath includes an entry port and a lumen to deliver irrigation fluid to the site of ablation. The handle of the catheter includes an electrical connection to control for electrical mapping and ablation. FIG. 1B shows more detail of the handle for the catheter. As shown in FIG. 1C, the jaws in this device are made of a single piece of flexible material that is biased in the open position. As shown in FIG. 1D, the jaws may include mapping electrodes in addition to ablation electrodes. The jaws also may or may not include ports for delivery of irrigation fluids. Use of the device is shown in FIG. 1E, when movement of the sheath distally results in closure of the jaws.

### EXAMPLE 2

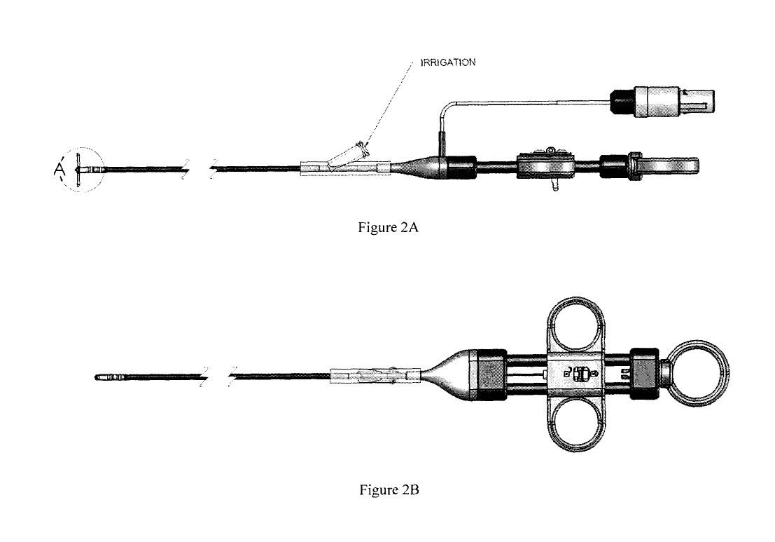

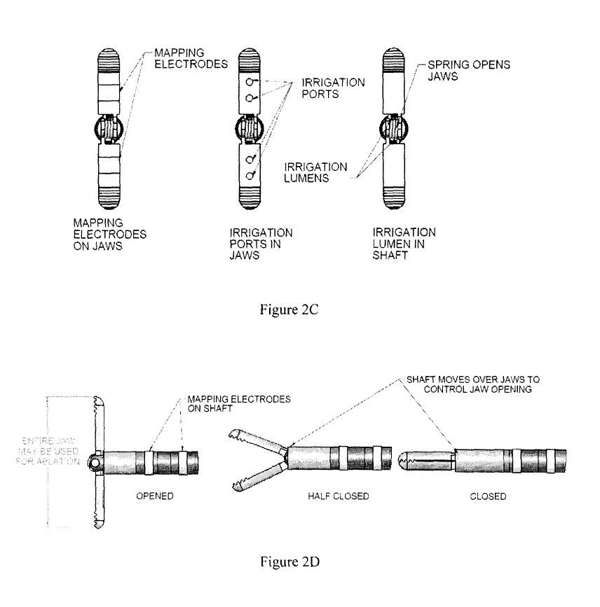

Another device of the invention is shown in FIGS. 2A-2D. FIGS. 2A-2B show generally the same configuration of the catheter and sheath as in Example 1. The jaws in this device are biased open by use of a spring. As with the device of Example 1, this device may include mapping electrodes and/or irrigation delivery ports in the jaws. In FIG. 2D, movement of the sheath distally results in closure of the jaws. FIG. 2D also illustrates how mapping electrodes may be disposed on the sheath.

### EXAMPLE 3

FIGS. 3A-3C illustrate use of a device of the invention. In FIG. 3A, the jaws in an open state are pressed against a tissue for mapping. In FIG. 3B, the sheath is moved distally to close the jaws and grasp the tissue. In FIG. 3C, the grasped tissue is ablated.

### OTHER EMBODIMENTS

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A device for tissue ablation comprising:
a catheter having a proximal end and a distal end;
a pair of jaws comprising an ablation electrode disposed at the distal end of the catheter; and
a sheath movable with respect to the catheter,
wherein, when the sheath is disposed proximally relative to the proximal end of the catheter, the pair of jaws is biased to an open position, and, when the sheath is disposed distally relative to the proximal end of the catheter, the pair of jaws is biased to a closed position.

2. The device of claim 1, wherein at least one of the catheter and sheath further comprises an irrigation lumen and a port configured and arranged to release irrigating fluid at the distal end of the catheter.

3. The device of claim 2, wherein the port is disposed in the pair of jaws.

4. The device of claim 2, wherein the port is disposed proximal to the pair of jaws.

5. The device of claim 1, wherein the sheath further comprises mapping electrodes.

6. The device of claim 1, wherein the pair of jaws further comprises mapping electrodes.

7. The device of claim 1, wherein the pair of jaws is the ablation electrode.

8. The device of claim 1, further comprising a mechanical element configured and arranged to bias the pair of jaws open when the sheath is disposed proximally.

9. The device of claim 8, wherein the mechanical element is a spring.

10. The device of claim 1, wherein the pair of jaws is shaped to be biased open when the sheath is disposed proximally.

11. The device of claim 10, wherein the pair of jaws is shaped from a single piece of flexible material.

12. The device of claim 1, wherein the pair of jaws further comprises a return electrode.

13. The device of claim 1, wherein the distal end of said catheter is deflectable.

14. The device of claim 1, wherein the distal end of said sheath is deflectable.

15. The device of claim 1, further comprising a lock configured and arranged to maintain the jaws in a closed state when the sheath is disposed proximally, and to release the jaws when the sheath is disposed distally.

16. A method of ablating a tissue in a subject, said method comprising the steps of:
inserting a device into the subject, the device including:
a catheter having a proximal end and a distal end,
a pair of jaws having an ablation electrode disposed at the distal end of the catheter, and
a sheath movable with respect to the catheter;
placing the pair of jaws adjacent to a tissue of interest in the subject;
actuating the pair of jaws and grasping the tissue of interest by extending the sheath away from the proximal end of the catheter; and
actuating the ablation electrode to ablate the tissue.

17. The method of claim 16, wherein the tissue of interest is along a ridge of a cardiac structure.

18. The method of claim 16, wherein actuating the pair of jaws includes moving the sheath by:
biasing the pair of jaws to an open position when the sheath is disposed proximally relative to the tissue, and
biasing the pair of jaws to a closed position when the sheath is disposed distally, relative to the tissue.

19. The device of claim 1, wherein the sheath is configured and arranged to move the jaws from the open position to the closed position by extending over at least a portion of the jaws and by exerting force that closes the jaws.

20. The device of claim 1, wherein
the sheath extends over at least a portion of the jaws when the sheath is disposed distally, and
about none of the sheath extends over the jaws when the sheath is disposed proximally.
